(19) United States
(12) Patent Application Publication
(10) Pub. No.: US 2025/0281104 A1
(43) Pub. Date: Sep. 11, 2025

(54) ARTIFICIAL INTELLIGENCE BASED NEONATAL SEIZURE DETECTION DEVICE, SYSTEM AND METHOD

(71) Applicant: Rekovar Inc., Irvine, CA (US)

(72) Inventor: Shiva Sharareh, Laguna Niguel, CA (US)

(21) Appl. No.: 19/075,561

(22) Filed: Mar. 10, 2025

Related U.S. Application Data
(60) Provisional application No. 63/563,163, filed on Mar. 8, 2024.

(51) Int. Cl. A61B 5/00; A61B 5/0205; A61B 5/1455; A61B 5/372

(52) U.S. Cl. CPC A61B 5/4094; A61B 5/0205; A61B 5/14551; A61B 5/372; A61B 5/6803; A61B 5/7203; A61B 5/7257; A61B 5/7267; A61B 5/7282; A61B 5/7289; A61B 5/742; A61B 2503/045

(57) ABSTRACT

Disclosed herein is a system and method for automatically detecting neonatal seizures. An example system comprises a headband enclosing a sensor placed on a neonate's head to monitor brain activity of the neonate, and a processor configured to obtain electroencephalogram (EEG) signals measured by the sensor. The system also comprises a computing device configured to: obtain the EEG signals from the wearable computing device, process the EEG signals into a plurality of frequency bands, incorporate and train a neural network to detect a first type of data anomaly in one or more segments of the EEG signals in each of the plurality of frequency bands, use the neural network to determine a second type of data anomaly the EEG signals based upon a correlation between observed patterns and known neonatal seizure patterns, and determine whether the EEG signals represents a seizure based upon the first and second anomalies.

### CROSS REFERENCE TO RELATED APPLICATION

The application claims priority to U.S. Provisional Patent Application No. 63/563,163, filed on Mar. 8, 2024, entitled “ARTIFICIAL INTELLIGENCE BASED NEONATAL SEIZURE DETECTION DEVICE, SYSTEM AND METHOD,” the content of which is incorporated by reference herein in its entirety.

### FIELD OF TECHNOLOGY

The present disclosure generally relates to artificial intelligence based seizure detection devices, systems and methods, and more particularly relates to using non-invasive electroencephalogram (EEG) wearable devices to continuously monitor and analyze brain activities of neonates using advanced machine learning and deep learning techniques.

### BACKGROUND

In recent years, neonatal abstinence syndrome (NAS) has emerged as a significant challenge in neonatal care, driven largely by the increasing prevalence of opioid use during pregnancy. NAS, a withdrawal syndrome in newborns following exposure to addictive substances in utero, often leads to a range of health complications, including neonatal seizures. These seizures are not only difficult to diagnose due to the subtlety of their clinical manifestations but also critical to manage promptly to mitigate long-term neurological damage.

EEG is a medical test that measures and records the electrical activity of the brain. It is a non-invasive procedure that involves placing electrodes on the scalp to detect the electrical impulses generated by neurons (brain cells). These electrodes are connected to an EEG machine, which amplifies and records the brain's electrical activity as wave patterns. EEG is commonly used to diagnose and monitor various neurological conditions, such as epilepsy, sleep disorders, and brain injuries. The recorded brainwave patterns can provide valuable information about brain function, including the presence of abnormal activity or patterns associated with specific neurological disorders. The EEG recording typically displays different types of brain waves, such as alpha, beta, delta, and theta waves, each associated with different states of consciousness and activities. In neonates with NAS, EEG may be useful for monitoring brain activity and seizures.

There are a number of design considerations necessary for precise data collection in wearable EEG neuro-monitoring devices, specifically tailored for use in the Neonatal Intensive Care Unit (NICU). One aspect involves the development of a form factor that is not only functional but also comfortable and safe for the delicate population it serves. Traditional EEG wearable devices often tend to be bulky and are typically constructed from rigid, hard plastics. There is a growing need for a paradigm shift towards more infant-friendly materials and designs. This necessity arises from the paramount importance of continuous, non-invasive monitoring in neonates, particularly for those at risk of neurological disorders. For example, these EEG wearable devices may need to be miniaturized and softened, ensuring they meet the unique requirements of neonatal care, such as the need for a gentle, yet secure fit on a small, sensitive head, and the ability to accommodate the rapid growth and delicate skin of newborns. Additionally, other potential applications of such wearable EEG technology may require extending its utility beyond the NICU to broader pediatric care and potentially to adult patients requiring similar non-invasive neurological monitoring.

Existing EEG wearable devices are predominantly tailored for adult use, with a specific focus on meditation and mindfulness practices. These devices, however, are not engineered for continuous wear, particularly during sleep or in positions that prioritize comfort. As such, directly miniaturizing these existing designs for neonatal use in the NICU presents significant safety concerns. Neonates, with their delicate physiology and unique needs, require a design that is fundamentally different-one that prioritizes safety and comfort for continuous, long-term wear.

Accordingly, there is a need for an EEG wearable device that not only accommodates the small size and sensitivity of infants but also ensures a secure, gentle fit that can be safely maintained during various states of rest and activity.

Furthermore, it is desirable to use non-invasive EEG wearable devices to continuously monitor the brain activity of neonates, providing real-time data that is essential for the early detection and accurate diagnosis of seizures. Current neonatal seizure detection methods and algorithms have modest sensitivity and relatively high false positive rates. There is a need for accurately and reliably detecting seizure activity using EEG signals on neonates.

### SUMMARY

Among other features, the present disclosure relates to a system deployed within a communication network for automatically detecting neonatal seizures. The system may comprise a wearable computing device, comprising: a headband enclosing at least one sensor placed on a selected location of a neonate's head to monitor brain activity of the neonate, and a first processor configured to obtain electroencephalogram (EEG) signals measured by the at least one sensor. The system may also comprise a computing device, comprising: a non-transitory computer-readable storage medium storing instructions, and a second processor coupled to the non-transitory computer-readable storage medium and configured to execute the instructions to: obtain the EEG signals from the wearable computing device, process the EEG signals into a plurality of frequency bands, incorporate and train a neural network to detect a first type of data anomaly in one or more segments of the EEG signals in each of the plurality of frequency bands, use the neural network to determine a second type of data anomaly in the one or more segments of the EEG signals based at least upon a correlation between a pattern of each of the one or more segments of the EEG signals and a plurality of waveform patterns of neonatal seizure patterns, and determine whether each of the one or more segments of the EEG signals represents a seizure based at least upon the first and second anomalies.

In one embodiment, the processor of the computing device may be further configured to execute the instructions to calculate a score to quantify a likelihood of the one or more segments of the EEG signals represent a seizure of the neonate, and display the score via a graphical user interface of the computing device.

In another embodiment, the processor of the computing device may be configured to execute the instructions to communicate with the wearable computing device via Bluetooth Low Energy (BLE) protocols.

In yet another embodiment, the processor of the computing device may be configured to execute the instructions to process the EEG signals into the plurality of frequency bands using fast Fourier transform and normalize processed EEG signals to have a uniform scale.

According to some embodiment, the system may further comprise a computing server deployed within the communication network and configured to host the neural network, wherein the neural network includes a Convolutional Long Short-Term Memory (ConvLSTM) model.

In some embodiments, the first type of data anomaly may include sharp waves and spikes in one or more segments of the EEG signals in each of the plurality of frequency bands, and the second type of data anomaly may include unusual EEG signal patterns indicating a seizure event due to a temporal context they appear in.

Moreover, the headband of the wearable computing device may further enclose an accelerometer configured to track and analyze movements of the neonate, and a pulse oximeter configured to detect oxygen saturation and respiratory pattern changes associated with seizures in the neonate. The processor of the computing device may be further configured to execute the instructions to receive accelerometer and pulse oximeter data from the wearable computing device, and determine whether each of the one or more segments of the EEG signals represents the seizure based upon the accelerometer and pulse oximeter data.

In accordance with other aspects, the present disclosure may relate to a computer-implemented method, comprising: measuring, by at least one sensor and a first processor of a wearable computing device, EEG signals of a neonate; obtaining, by a computing device, the EEG signals from the wearable computing device; processing the EEG signals into a plurality of frequency bands; incorporating and training a neural network to detect a first type of data anomaly in one or more segments of the EEG signals in each of the plurality of frequency bands; using the neural network to determine a second type of data anomaly in the one or more segments of the EEG signals based at least upon a correlation between a pattern of each of the one or more segments of the EEG signals and a plurality of waveform patterns of neonatal seizure patterns; and determining whether each of the one or more segments of the EEG signals represents a seizure based at least upon the first and second anomalies.

In an embodiment, the computer-implemented method may further comprise calculating a score to quantify a likelihood of the one or more segments of the EEG signals represent a seizure of the neonate; displaying the score via a graphical user interface of the computing device; and communicating, by the computing device, with the wearable computing device via BLE protocols.

According to some implementations, the processing the EEG signals into the plurality of frequency bands may comprise using fast Fourier transform to process the EEG signals in each of the plurality of frequency bands and normalizing processed EEG signals to have a uniform scale.

Furthermore, the computer-implemented method may comprise hosting, by a computing server deployed within the communication network, the neural network, wherein the neural network includes a ConvLSTM model.

According to some implementations, the first type of data anomaly may include sharp waves and spikes in one or more segments of the EEG signals in each of the plurality of frequency bands, and the second type of data anomaly may include unusual EEG signal patterns indicating a seizure event due to a temporal context they appear in.

In addition, the computer-implemented method may comprise tracking and analyzing movements of the neonate using an accelerometer enclosed in the headband of the wearable computing device; detecting oxygen saturation and respiratory pattern changes associated with seizures in the neonate using a pulse oximeter; receiving, by the computing device, accelerometer and pulse oximeter data from the wearable computing device; and determining whether each of the one or more segments of the EEG signals represents the seizure based upon the accelerometer and pulse oximeter data.

In accordance with additional aspects, the present disclosure relates to a non-transitory computer readable medium storing machine executable instructions for a system deployed within a communication network for automatically detecting neonatal seizures, the machine executable instructions being configured for: measuring, by at least one sensor and a first processor of a wearable computing device, EEG signals of a neonate; obtaining, by a computing device, the EEG signals from the wearable computing device; processing the EEG signals into a plurality of frequency bands; incorporating and training a neural network to detect a first type of data anomaly in one or more segments of the EEG signals in each of the plurality of frequency bands; using the neural network to determine a second type of data anomaly in the one or more segments of the EEG signals based at least upon a correlation between a pattern of each of the one or more segments of the EEG signals and a plurality of waveform patterns of neonatal seizure patterns; and determining whether each of the one or more segments of the EEG signals represents a seizure based at least upon the first and second anomalies.

In an embodiment, the non-transitory computer readable medium may further comprise instructions for: calculating a score to quantify a likelihood of the one or more segments of the EEG signals represent a seizure of the neonate; and displaying the score via a graphical user interface of the computing device.

Moreover, the non-transitory computer readable medium may further comprise instructions for communicating, by the computing device, with the wearable computing device via BLE protocols.

According to some implementations, the instructions for processing the EEG signals into the plurality of frequency bands may comprise instructions for using fast Fourier transform to process the EEG signals in each of the plurality of frequency bands and normalizing processed EEG signals to have a uniform scale.

Additionally, the non-transitory computer readable medium may comprise instructions for: hosting, by a computing server deployed within the communication network, the neural network, wherein the neural network includes a ConvLSTM model; tracking and analyzing movements of the neonate using an accelerometer enclosed in the headband of the wearable computing device; detecting oxygen saturation and respiratory pattern changes associated with seizures in the neonate using a pulse oximeter; receiving, by the computing device, accelerometer data from the wearable computing device; and determining whether each of the one or more segments of the EEG signals represents the seizure based upon the accelerometer data.

Further, the first type of data anomaly may include sharp waves and spikes in one or more segments of the EEG signals in each of the plurality of frequency bands, and the second type of data anomaly may include unusual EEG signal patterns indicating a seizure event due to a temporal context they appear in.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplary pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 illustrates a computing system deployed within a computing environment and communication network for monitoring and analyzing brain activity of neonates using advanced machine learning and deep learning techniques, according to an exemplary aspect of the present disclosure;

FIG. 2 illustrates a block diagram illustrating various components and modules of a wearable EEG device, a computing device, and a backend computing server system of the computing system of FIG. 1, according to an exemplary aspect of the present disclosure;

FIG. 3 illustrates a front view of a first design of an EEG band having a plurality of electrodes, according to an exemplary aspect of the present disclosure;

FIG. 4 illustrates a front view of a second design of an EEG band having a plurality of electrodes, according to an exemplary aspect of the present disclosure;

FIG. 5(A) illustrates a front view of a first embodiment of an EEG band having a plurality of electrodes, according to an exemplary aspect of the present disclosure;

FIG. 5(B) illustrates a front view of a second embodiment of an EEG band having a plurality of electrodes, according to an exemplary aspect of the present disclosure;

FIG. 5(C) illustrates a front view of a third embodiment of an EEG band having a plurality of electrodes, according to an exemplary aspect of the present disclosure;

FIG. 6 illustrates an example neural network architecture, according to an exemplary aspect of the present disclosure;

FIG. 7 illustrates a graphical user interface showing EEG data collection and analysis results, according to an exemplary aspect of the present disclosure; and

FIG. 8 illustrates a flowchart of a method for monitoring and analyzing brain activity of neonates using advanced machine learning and deep learning techniques, according to an exemplary aspect of the present disclosure.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

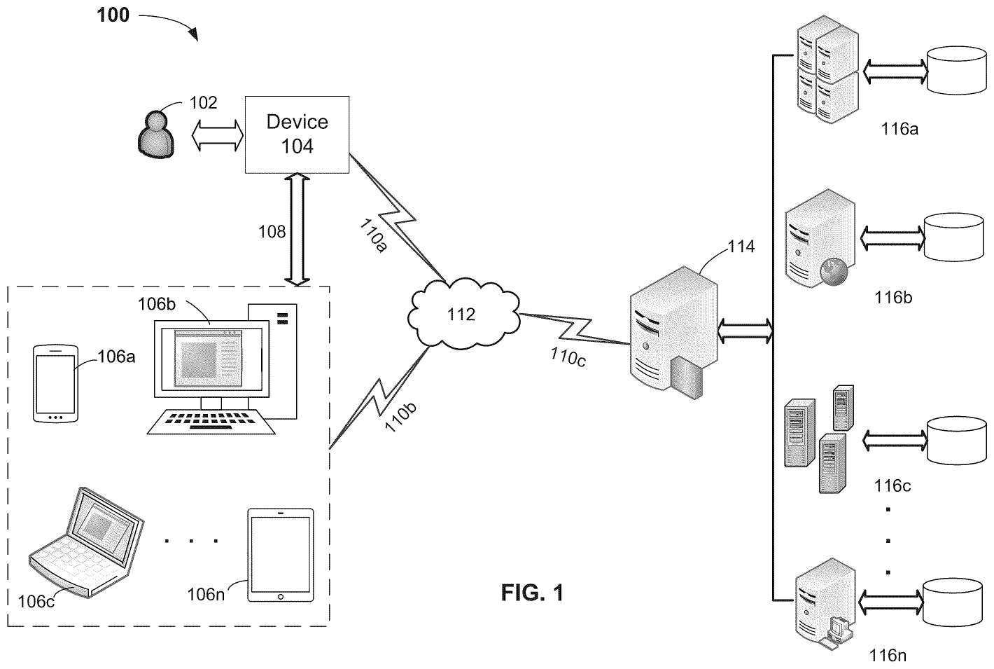

Referring to FIG. 1, in accordance with aspects of the present disclosure, a computing system 100 deployed within a computing environment and communication network may be configured to record the electrical activity of a patient 102's brain via an EEG wearable device 104, identify anomalies in obtained EEG signals using at least one computing device 106a, 106b, 106c . . . 106n and/or a backend computing server 114 based on advanced machine learning/deep learning techniques and artificial intelligence models, and detect seizures based on the identified anomalies in the EEG signals.

In one aspect, patient 102 may be a neonate suffering from or prone to a neurological condition called neonatal seizures, which are defined as the occurrence of sudden, paroxysmal, abnormal alteration of electrographic activity at any point from birth to the end of the neonatal period (the first four weeks of a child's life). During this period, because the neonatal brain is developmentally immature, neonatal seizures have unique pathophysiology and electrographic findings resulting in clinical manifestations that can be different and more difficult to identify when compared to older age groups. The prognosis of neonatal seizures depends on the underlying etiology. For example, if the EEG of a neonate has abnormalities, such a neonate may have a poor prognosis and may develop cerebral palsy and epilepsy. The presence of spikes on EEG may have an elevated risk of developing future epilepsy. The mortality rate of neonatal seizures has been reported to be high. In survivors, neurologic impairment, disability, developmental delay, and epilepsy are common. As will be described fully below, an EEG wearable device 104 may be fitted to the head of the patient 102 to record electrical signals of the brain in order to identify abnormal brain activity or an event associated with seizures.

It should be appreciated that the wearable EEG technology of the present disclosure may be extended to broader pediatric care and potential adult patients where similar non-invasive neurological monitoring is desired, such as the need for a miniaturized, softened, and secure fit on the head, and the ability to accommodate the rapid growth and delicate and sensitive skin. In alternate embodiments, the patient 102 may be an animal (e.g., a rat, a cat, a monkey, a dog, a cow, or any mammals).

In one aspect, the EEG device 104 may be configured to transmit the EEG signals of the patient 102 to a selected computing device or system (e.g., one of 106a, 106b, 106c . . . 106n) via a wireless communication protocol 108 (e.g., Bluetooth, Bluetooth Low Energy (BLE), Wi-Fi). The EEG device 104 may also transmit signals to the backend computing server 114 via a network 112 and communication protocols 110a, 110c.

According to one embodiment, an application, which may be a mobile or web-based application (e.g., native iOS or Android Apps), may be downloaded and installed on the selected computing device or system 106a, 106b, 106c, . . . 106n for instantiating a seizure detection module, and interacting with a user of the application, among other features. For example, such an application may be used by caregivers of patient 102, medical professionals, neonatal seizure researchers, EEG technicians or technologists, trained staff (e.g., nurses, support staff, monitor technicians), and other end-users. Automated agents, scripts, playback software, and the like acting on behalf of one or more people may also be users. Such a user-facing application of the system 100 may include a plurality of modules executed and controlled by the processor of the hosting computing device or system 106a, 106b, 106c, 106n for processing EEG signals received from the device 104 using various seizure detection algorithms, as will be described fully below. Computing device 106a, 106b, 106c, . . . 106n hosting the mobile or web-based application may be configured to connect, using a suitable communication protocols 110b, 110c and network 112, with the backend computing server 114. Here, communication network 112 may generally include a geographically distributed collection of computing devices or data points interconnected by communication links and segments for transporting signals and data therebetween. Communication protocol(s) 110a, 110b, 110c may generally include a set of rules defining how computing devices and networks may interact with each other, such as frame relay, Internet protocol (IP), transmission control protocol (TCP), user datagram protocol (UDP), hypertext transfer protocol (HTTP). It should be appreciated that the system 100 of the present disclosure may use any suitable communication network, ranging from local area networks (LANs), wide area networks (WANs), cellular networks, to overlay networks and software-defined networks (SDNs), a packet data network (e.g., the Internet), mobile telephone networks (e.g., cellular networks, such as 4G or 5G), Plain Old Telephone (POTS) networks, and wireless data networks (e.g., Institute of Electrical and Electronics Engineers (IEEE) 802.11 family of standards known as Wi-Fi®, WiGig®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, virtual private networks (VPN), Bluetooth, Near Field Communication (NFC), or any other suitable network.

In some embodiments, the backend computing server 114 may be Cloud-based or an on-site server. The term “server” generally refers to a computing device or system, including processing hardware and process space(s), an associated storage medium such as a memory device or database, and, in some instances, at least one database application as is well known in the art. The backend computing server system 114 may provide functionalities for any connected devices such as sharing data or provisioning resources among multiple client devices, or performing computations for each connected client device. According to a preferred embodiment, within a Cloud-based computing architecture, the backend computing server 114 may provide various Cloud computing services using shared resources. Cloud computing may generally include Internet-based computing in which computing resources are dynamically provisioned and allocated to each connected computing device or other devices on-demand, from a collection of resources available via the network or the Cloud. Cloud computing resources may include any type of resource, such as computing, storage, and networking. For instance, resources may include service devices (firewalls, deep packet inspectors, traffic monitors, load balancers, etc.), computing/processing devices (servers, central processing units (CPUs), graphics processing units (GPUs), random access memory, caches, etc.), and storage devices (e.g., network attached storages, storage area network devices, hard disk drives, solid-state devices, etc.). In addition, such resources may be used to support virtual networks, virtual machines, databases, applications, etc. The term “database,” as used herein, may refer to a database (e.g., relational database management system (RDBMS) or structured query language (SQL) database), or may refer to any other data structure, such as, for example a comma separated values (CSV), tab-separated values (TSV), JavaScript Object Notation (JSON), extendible markup language (XML), TeXT (TXT) file, flat file, spreadsheet file, and/or any other widely used or proprietary format. In some embodiments, one or more of the databases or data sources may be implemented using one of relational databases, flat file databases, entity-relationship databases, object-oriented databases, hierarchical databases, network databases, NoSQL databases, and/or record-based databases.

Cloud computing resources accessible using any suitable communication network (e.g., Internet) may include a private Cloud, a public Cloud, and/or a hybrid Cloud. Here, a private Cloud may be a Cloud infrastructure operated by an enterprise for use by the enterprise, while a public Cloud may refer to a Cloud infrastructure that provides services and resources over a network for public use. In a hybrid Cloud computing environment which uses a mix of on-premises, private Cloud and third-party, public Cloud services with orchestration between the two platforms, data and applications may move between private and public Clouds for greater flexibility and more deployment options. Some example public Cloud service providers may include Amazon (e.g., Amazon Web Services® (AWS)), IBM (e.g., IBM Cloud), Google (e.g., Google Cloud Platform), and Microsoft (e.g., Microsoft Azure®). These providers provide Cloud services using computing and storage infrastructures at their respective data centers and access thereto is generally available via the Internet. Some Cloud service providers (e.g., Amazon AWS Direct Connect, Microsoft Azure ExpressRoute) may offer direct connect services and such connections typically require users to purchase or lease a private connection to a peering point offered by these Cloud providers.

In certain implementations, the backend computing server 114 (e.g., Cloud-based or an on-site server) of the present disclosure may be configured to connect with various data sources or services 116a, 116b, 116c, . . . 116n. As will be described fully below, the backend computing server system 114 may be configured to host, train, operate, and/or incorporate any type of artificial intelligence model (e.g., at least one of 116a, 116b, 116c, . . . 116n) for predicting and detecting neonatal seizure events based at least upon EEG signals obtained from patient 102. Example models may include but not limited to a neural network, a convolutional neural network (CNN), a recurrent neural network (RNN), a deep neural network (DNN), a decision tree, a support vector machine (SVM), a regression, and/or a Bayesian network.

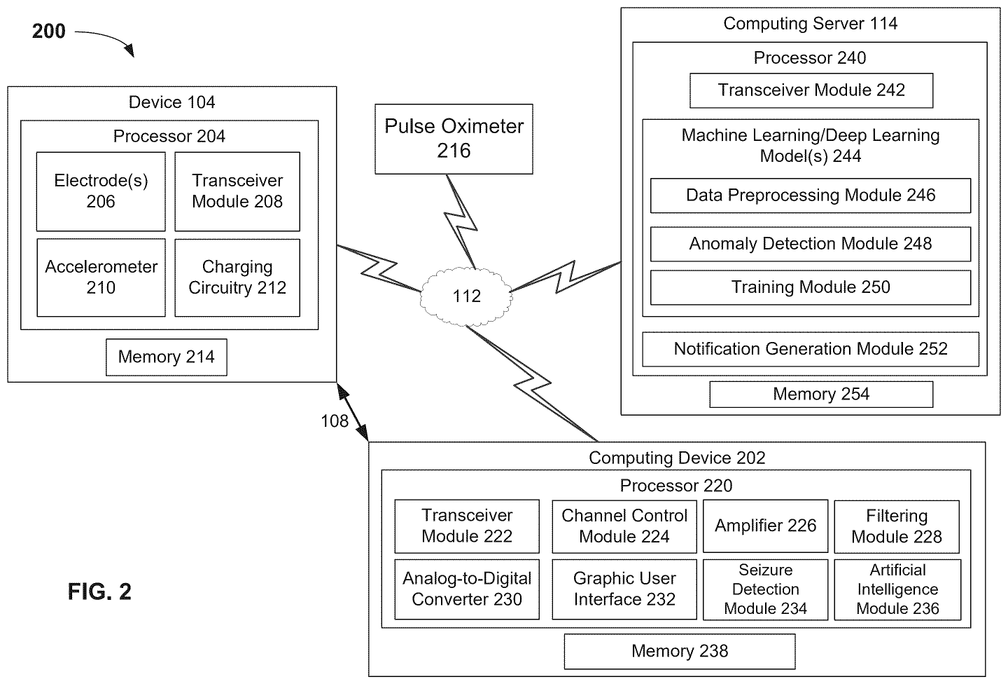

FIG. 2 is a block diagram 200 illustrating various components and modules of the device 104, a computing device 202 (e.g., one of computing device 106a, 106b, 106c . . . 106n), and the backend computing server 114, in accordance with aspects of the present disclosure.

In a preferred embodiment, the device 104 may be a non-invasive, reliable, and efficient EEG headband configured to accurately detect seizures, particularly in infants. Early detection of seizure events can lead to timely intervention, improving the overall prognosis and quality of life for these young patients. To ensure maximum comfort, the EEG headband of the present disclosure is designed to be as small and lightweight as possible, tailored to fit an infant's head without causing any discomfort or hindrance. Furthermore, the design of the present disclosure prioritizes energy efficiency, aiming for a selected period of time (e.g., at least 12 hours) of continuous operation, thus providing caregivers and medical professionals with extended monitoring capabilities. Additionally, the design being affordable is essential to make the EEG headband accessible to a wider population, thereby promoting its widespread adoption and ultimately contributing to better seizure management and improved healthcare outcomes for infants.

A seizure is a brief episode involving changes in consciousness and/or involuntary shaking or jerking of the body. Seizure phases generally include an aural stage, an ictal stage, and a postictal stage. The first stage of a seizure, an aura, is also described as the pre-ictal phase, can last from a few seconds to an hour in duration, which may be characterized by a spike in brain activity that can be detected using an EEG. Specifically, when a neuron receives enough excitatory signals from sensory cells and other neurons, it produces a response called an action potential, which causes the neuron to release chemicals that excite all cells connected to a part of the firing neuron called the axon. During this process, there is a rapid exchange of ions (electrically-charged particles) that changes the voltage of the fluid surrounding the firing neuron in a predictable fashion. This voltage change may travel spherically outward from the firing neuron until it reaches the patient 102's skull. The EEG headband of the present disclosure may be configured to record brain activity by detecting voltages at one or more scalp locations over time, which alter in response to the firing of many neurons simultaneously, using one or more electrodes attached to the surface of the head of the patient 102. Voltage signals may be sampled from the electrodes at high frequencies (e.g., 1 kHz to 2 kHz) to provide an effectively continuous stream of data known as an EEG waveform. According to one implementation, spectral information may be extracted from the EEG waveform, which results in discrete frequency-band ratios (wave types) generated from the raw data at frequencies around 100 Hz. These are generally divided into, e.g., delta, theta, alpha, beta, and gamma waves, with each type representing a specific range of frequencies.

As will be described fully below, EEG signals obtained from device 104 may be analyzed for diagnosing and monitoring epilepsy and performing seizure prediction with artificial intelligence technology. During an epileptic seizure, the EEG can accurately detect an episode even in the absence of physical signs of patient 102. This is because brain activity during a seizure produces characteristic patterns that are distinct from normal brain activity. These patterns can be observed based on the EEG signals and can help to identify the type of seizure and its location in the brain. Furthermore, the EEG signals can also provide information about the progression of the seizure and its aftermath. After the seizure, there is a period of postictal activity where the brain is recovering from the seizure. This is also characterized by distinct patterns on the EEG, which can help to determine the severity of the seizure and the potential risk of further seizures.

In one embodiment, as shown in FIG. 2, a microcontroller, processor, or graphics processing unit (hereinafter “processor”) 204 of the device 104 may be configured to control and execute a plurality of modules which may include electrode(s) 206, a transceiver module 208, an accelerometer 210, and charging circuitry 212. The term “module” as used herein refers to a real-world device, component, or arrangement of components and circuitries implemented using hardware, such as by an application specific integrated circuit (ASIC) or field-programmable gate array (FPGA), for example, or as a combination of hardware and software, such as by e.g., the processor 204 and a set of instructions to implement the module's functionality, which (while being executed) transform the microcontroller into a special purpose device. A module may also be implemented as a combination of the two, with certain functions facilitated by hardware alone, and other functions facilitated by a combination of hardware and software. Each module may be realized in a variety of suitable configurations, and should not be limited to any example implementation exemplified herein.

Memory 214, which is coupled to processor 204, may be configured to store at least a portion of information obtained by the device 104. In one aspect, memory 214 may be a non-transitory machine-readable medium configured to store at least one set of data structures or instructions (e.g., software) embodying or utilized by at least one of the techniques or functions described herein. It should be appreciated that the term “non-transitory machine-readable medium” may include a single medium or multiple media (e.g., one or more caches) configured to store at least one instruction. The term “machine readable medium” may include any medium that is capable of storing, encoding, or carrying instructions for execution by all modules of the device 104 and that cause these modules to perform at least one of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks.

According to one implementation, device 104 may use a single electrode or a single EEG channel for seizure detection. Seizures typically involve synchronous neuronal firing, causing strong and easily identifiable patterns in the recorded electrical signals. By placing a single EEG channel in a strategic location on the scalp of patient 102, the EEG headband of the present disclosure can capture this distinctive electrical activity of patient 102's brain, which tends to propagate across multiple brain regions. Furthermore, the computing device 202 and/or the backend computing server 114 may be configured to use advanced signal processing and machine learning algorithms to extract relevant features and patterns from detected electrical signals, even when captured by a single electrode. In one aspect, a single electrode design of the present disclosure may be sufficient for detecting seizure activity while reducing the size of the EEG wearable device. This also allows the device 104 to be a less invasive and more cost-effective solution. It should be appreciated that the present disclosure may be configured to use multiple electrodes, which may provide more spatial information and potentially enhance detection accuracy, as brainwave activity is captured from different brain regions at the same time.

The transceiver module 208 of device 104 may be configured by processor 204 to exchange various information and data with other computing devices deployed with the communication network 112. According to one preferred embodiment, the transceiver module 208 may be configured to facilitate BLE connectivity, enhancing the EEG wearable device's wireless communication capabilities. For example, BLE connectivity allows for seamless, real-time data transmission to healthcare professionals, enabling prompt and informed medical decisions. Further, device 104 may achieve low power consumption using the BLE connectivity.

In yet another aspect, the integration of at least one accelerometer 210 allows the EEG wearable device to track and analyze the infant patient 102's movements, providing vital information that can be correlated with EEG data. This feature is particularly important in the context of NAS, where monitoring the physical symptoms alongside the neurological ones can offer a more comprehensive understanding of the infant's condition. The accelerometer 210's data, when combined with the EEG readings, offers a multifaceted view of the neonate's health, potentially revealing subtle seizure activity that might not be solely detectable through EEG signals.

In some implementations, device 104 may include charging circuitry 212 for power management, making the device reusable, and in compliance with relevant electromagnetic interference and compatibility testing. In one example, the power source of the device 104 may include a compact sized battery. In addition, the circuitry design of the device 104 has a focus on miniaturization and efficiency, and the layout has been optimized to maximize space utilization while maintaining functionality. The grounding scheme for various signal paths has been selected and tested to minimize noise and interference.

In certain aspects, the development of the EEG headband is based on the following design criteria: (1) wearability, (2) accuracy, and (3) ease of manufacturing. The wearability and accuracy of the device design are equally important, because the headband is intended for extended use and data collected will contribute to the doctor's care. For the wearability of the device, the materials need to be soft and comfortable. The use of any bulky or rigid parts may be limited because that could irritate a baby's skin. The headband also needs to fit a range of head circumferences. For the accuracy of the device, it is imperative that the metal of the electrodes have sufficient physical contact with the baby's skin. Therefore, the headband must fit snuggly, but not tight. For the ease of manufacturing, a straightforward design decreases the product cost and will allow for more accessibility to those of different socio-economic backgrounds.

For the main fabric of the EEG headband, an elasticity may be selected such that the headband stretches to fit on a baby's head but would not slip off. Elasticity generally refers to the ability of a material to stretch and then return to its original shape and size when the stretching force is removed. Exemplary elastically stretchable materials may include stretch knit, polyester elastic fabric, Lycra®, elastane, Spandex, Spandex blends and the like. In order to account for a range of head circumferences, additional knit elastic and buckles may be used. In one embodiment, a fabric of the headband may be selected to have a 1.5× stretch (Lf/Li=1.5) for both the wearability as well as ease of manufacturing, and the simplified design may eliminate hard plastic and use less expensive materials and fewer components. Further, the headband may be designed to have a horizontal stretch but limited vertical stretch. That is, the headband may be designed to have a horizontal stretch factor greater than its vertical stretch factor.

To ensure the stability of the device and battery in the headband, in yet another embodiment, a piece of nylon webbing may be used as a rigid backing. This ensures that there are two layers of fabric and nylon webbing between the device and the baby's head. This significantly reduces the irritation that could arise when the baby is sleeping on its back.

According to certain aspects, as shown in FIG. 2, additional screening devices such as a pulse oximeter 216 may be used by the system 100 of the present disclosure to investigate oxygen saturation and respiratory pattern changes associated with EEG seizures in neonates. Pulse oximeter 216 may be configured to detect the amount of oxyhemoglobin and deoxygenated hemoglobin in arterial blood and shows it as oxyhemoglobin saturation (SₚO₂) which is an indirect estimation of arterial oxygen saturation (SₐO₂). The normal amount of SₚO₂ in healthy individuals is 97% to 99%. Pulse oximeter 216 may be attached to a patient's fingers, forehead, nose, foot, ears, or toes. In one embodiment, pulse oximetry by earlobe or forehead probes may have higher accuracy compared to other locations. The pulse oximeter 216 may be implemented as a separate device or integrated in the headband of the device 104. Data obtained from pulse oximeter 216 may be correlated with the EEG data to detect abrupt onset and clustering episodes of apnea and oxygen desaturation in term infants which may be a sign of epileptic seizures.

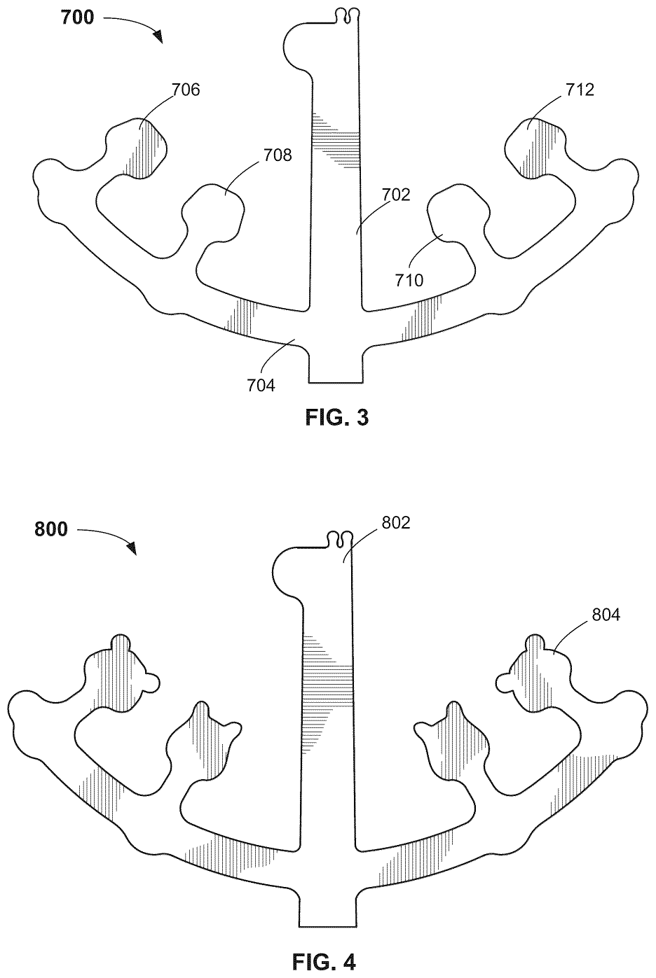

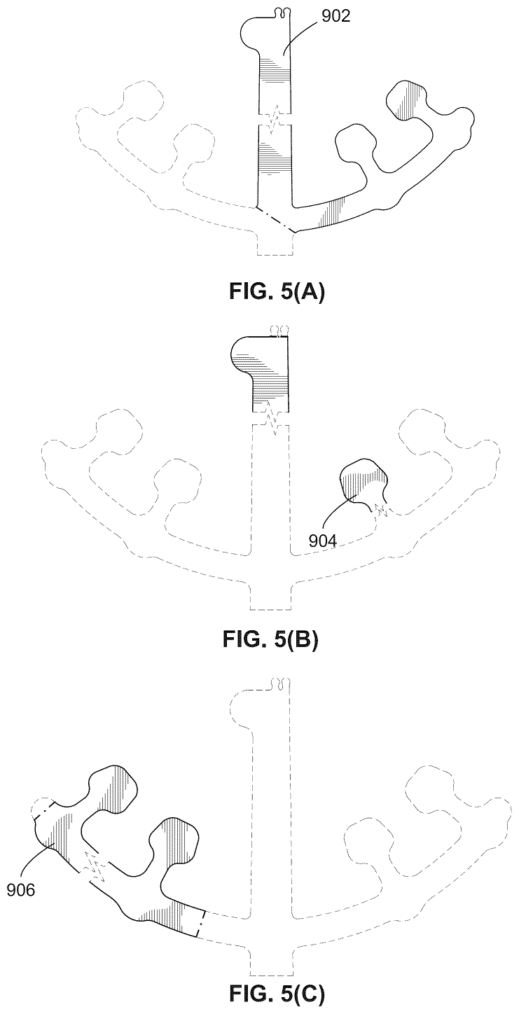

In accordance with aspects of the present disclosure, FIGS. 3 and 4 respectively illustrate a front view of two designs of an EEG band having a plurality of electrodes. For example, FIG. 3 illustrates a first design 700 of an EEG band having a central strip 702 connecting with a C-shaped or arched flexible band 704. One or more extensions 706, 708, 710, and 712 may be implemented along the inner circumference C-shaped flexible band 704. During use, the central strip 702 may be centrally positioned across the top of a patient's head, such that the extensions 706, 708, 710, and 712 are be positioned on left and right sides of the patient's head, respectively. Each of these extensions 706, 708, 710, and 712 may include an end portion to accommodate or fixed with an electrode. Referring to FIG. 4, a second design 800 of an EEG band may have a structure and configuration similar to that of the first design 700 of FIG. 3. In some embodiments, the end portion 802 of the central strip and the end portion 804 of each extension may have a shape different than that of the first design 700 of FIG. 3. FIG. 5(A) illustrates that an end portion 902 of the central strip may be removed or replaced with any appropriate parts. Similarly, FIG. 5(B) illustrates that an end portion 904 of a selected extension may be removed or replaced with any appropriate parts. Further, FIG. 5(C) illustrates that a selected extension 906 may be removed from the C-shaped flexible band or replaced with any appropriate parts. It should be appreciated that the specific structure, shape, dimensions and electrode array layout of the EEG band of the present disclosure may vary, depending upon each patient's conditions and EEG measurement requirements.

Referring back to FIG. 2, in accordance with aspects of the present disclosure, the device 104 may transmit EEG signals and accelerometer data and pulse oximeter 216's data to a selected computing device 202 (e.g., one of computing device 106a, 106b, 106c . . . 106n) which includes a processor 220 configured to control and execute a plurality of modules including but not limited to a transceiver module 222, a channel control module 224, a differential amplifier 226, a filtering module 228, an analog-to-digital converter (ADC) 230, a graphical user interface (GUI) 232, a seizure detection module 234, and an artificial intelligence module 236. Memory 238, which is coupled to the processor 220, may be configured to store at least a portion of information obtained by the computing device 202 and store at least one set of data structures or instructions (e.g., software) embodying or utilized by at least one of the techniques or functions described herein.

For example, the transceiver module 222 may be configured to communicate with the transceiver module 208 of the device 108 via any suitable wireless protocols such as wireless LAN, Wi-Fi, Bluetooth, ZigBee, Wi-Fi Direct, ultra-wideband, infrared data association (IrDA), BLE, and NFC. In one preferred embodiment, BLE connectivity 108 may be used between the two transceivers 208, 222 to exchange data in real-time.

Further, if the device 104 uses multiple electrodes and EEG channels, the computing device 202 may include a channel control module 224 configured to receive EEG signals from a particular electrode, or a portion of an array of electrodes, or all of the available electrodes in real-time. The scalp EEG signals of a patient may be recorded by different modes such as unipolar and bipolar modes. In a unipolar mode, the voltage differences between all electrodes and a reference one may be recorded, where a channel is formed by an electrode-reference pair. In the bipolar mode, the voltage differences between two specified electrodes are recorded, where each pair forms a channel. An electrode placement scheme on scalp, known as International 10-20 system, was recommended by the International Federation of Societies for Electroencephalography and Clinical Neurophysiology (IFSECN). The channel control module 224 may also determine which channel of the EEG signals is appropriate for seizure detection and analysis. For example, the channel selection may be based on patient-specific information such as patient's medical conditions.

Subsequently, computing device 202 may use a series of modules including amplifier 226, filtering module 228, and ADC 230 to respectively decompose the raw EEG signals into different frequency bands, and amplify and digitize the received EEG signals. Amplification of a comparatively tiny brain signal may bring it to a level where it can be used by the ADC 230. Filtering module 228 may process received EEG signals in real-time using any suitable low-pass, band-pass, or high-pass filters. In a preferred embodiment, one or more 6th order Butterworth filters may be used to decompose the EEG signals into e.g., delta (1 to 4 Hz), theta (4 to 8 Hz), alpha (8 to 12 Hz), beta (12 to 25 Hz), and gamma (25 to 140 Hz). Butterworth filters include a maximally flat magnitude response in the passband region, and a gain of 0 dB at direct current. The ADC 230 then converts the analog, continuous brain signal to a quantified, digital representation that may be further analyzed by computing device 202.

In one aspect, computing device 202 includes a web-based GUI 232 to process EEG digital signals. For example, the GUI 232 may be configured to transform obtained data into specific formats (e.g., CSV files) and instantiate different seizure detection algorithms utilized by seizure detection module 234 and/or artificial intelligence module 236 for analysis purposes. A first example algorithm utilized by seizure detection module 234 may process obtained EEG signals using selected processing techniques and produce a score based on the frequency and occurrence of brainwaves. A second example algorithm utilized by artificial intelligence module 236 may perform classification of brain signals through the application of machine learning/deep learning techniques. As a result, a score based on artificial intelligence technology may be calculated.

In some embodiments, the GUI 232 of the computing device 202 may be a thin client device/terminal/application deployed within the system 100 and may be configured to perform certain preliminary processing of raw EEG signals and information from various users. Thereafter, the processed data may be transmitted to e.g., machine learning/deep learning model(s) 244 of the computing server 114 to detect and predict seizure events, as will be described fully below. In one embodiment, the GUI 232 may include an application programming interface (API) interface configured to make one or more API calls therethrough. On the other hand, the computing server 114 may include an API gateway device (not shown) configured to receive and process API calls from various connected computing devices deployed within the system 100 (e.g., an operating system, a library, a device driver, an API, an application program, software or other module). Such an API gateway device may specify one or more functions, methods, classes, objects, protocols, data structures, formats and/or other features of the computing server 114 that may be used by the mobile or web-based application of the computing device 202. For example, the API interface may define at least one calling convention that specifies how a function associated with the computing server 114 receives data and parameters from a requesting device/system and how the function returns a result to the requesting device/system. It should be appreciated that the computing server 114 may include additional functions, methods, classes, data structures, and/or other features that are not specified through the API interface and are not available to a requesting computing device.

Seizures in the neonatal stage may only be detected by monitoring EEG signals. In some aspects, multiple approaches may be used to extract the signature of a seizure signal with computer algorithms, to mimic a clinician searching for repetitive pseudo-period waveforms/wave sequences and to use general data-driven EEG characteristics which involve extraction of features to capture information that form a description of an EEG signal. According to an embodiment, convolutional neural networks (CNNs) may be used by the computing device 202 and the computing server 114 to learn features from raw EEG data. The CNN may be compared to support vector machines (SVMs) as a baseline for performance, as SVMs have been validated and clinically accepted levels of accuracy. For example, system 100 of the present disclosure may use SVM as a baseline which has been validated on a clinical dataset with state-of-the-art seizure detection.

For SVM implementation, raw EEG data undergoes preprocessing of the signal and segmentation. A plurality of features may be selected (e.g., 55 features) and extracted from the preprocessed EEG and are normalized before the classification stage. A Gaussian kernel SVM may be trained on extracted features with per-channel seizure annotations. Five-fold cross-validation may be carried out to find the most optimized parameters to use on the training data. A moving average may be applied during the post-processing stage where the maximum probabilities are computed to represent the final probability.

Fully convolutional neural networks (FCNNs) have a similar approach to SVMs, as they have the same routines for preprocessing and post-processing EEG data. The difference is that raw EEG data may be used for the FCNN and not the extracted features for the SVM. In one embodiment, the network architecture of the present disclosure is as follows with six 1-dimensional convolutional filters constructed with Keras deep learning library. Convolution is followed by average pooling. The final convolutional layer is followed by global average pooling layers (GAPs) which output two probabilities for seizure and non-seizure probabilities. The CNN may be trained on categorical cross-entropy with stochastic gradient descent.

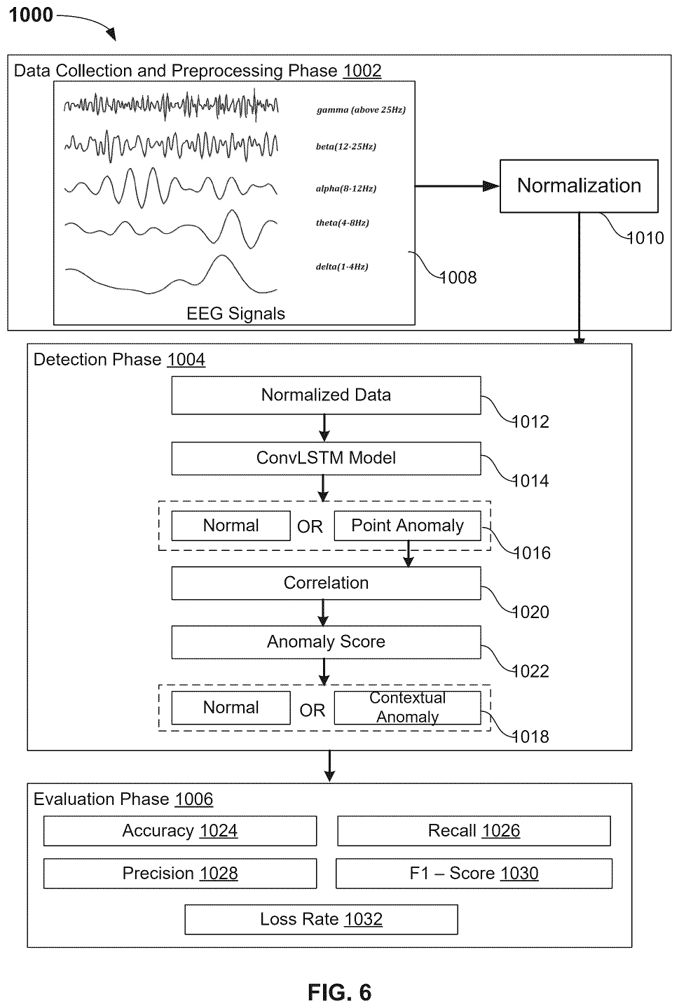

Referring now to FIGS. 2 and 6, in accordance with aspects of the present disclosure, an example architecture 1000 of the computing server 114 for seizure detection may be based on one or more machine learning models that leverage EEG data and other physiological and movement data (e.g., via accelerometer 210 and pulse oximeter 216) and obtained from either the device 104 or the computing device 202. In one implementation, the computing server 114 may include a processor 240 configured to control and execute a plurality of modules including but not limited to a transceiver module 242, machine learning/deep learning model(s) 244 which includes a data preprocessing module 246, an anomaly detection module 248 and a training module 250, and a notification generation module 252. Memory 254, which is coupled to the processor 240, may be configured to store at least a portion of information obtained by the computing server 114 and store at least one set of data structures or instructions (e.g., software) embodying or utilized by at least one of the techniques or functions described herein. Architecture 1000 of the computing server 114 may be generally divided into three main phases: a data collection and preprocessing phase 1002, a detection phase 1004, and an evaluation phase 1006. The goal is to accurately detect seizures by identifying anomalies in EEG signals.

According to one embodiment, data collection and preprocessing phase 1002 of the computing server 114 may begin with an EEG data acquisition operation. The first step may involve collecting, by the transceiver module 242, EEG data which reflects the brain's electrical activity from either device 104 or computing device 202 as illustrated in FIG. 2. This data is typically gathered from patients suspected of having seizure disorders using scalp electrodes, as described above. Before the EEG data is input into a machine learning model (e.g., a neural network), a filtering process may be carried out to extract the time-domain waveforms in a number of different frequency ranges 1008 (e.g., delta (1 to 4 Hz), theta (4 to 8 Hz), alpha (8 to 12 Hz), beta (12 to 25 Hz), and gamma (25 to 140 Hz)) using Fast Fourier Transform (FFT). For example, the filter module 228 of computing device 202 or a data preprocessing module 246 of the machine learning/deep learning model(s) 244 of the computing server 114 may be configured to perform such filtering and FFT. This process should be done before the neural network as FFT is a very computation efficient algorithm and will reduce computational complexity and save processing power in the neural network.

Raw EEG data is subject to a range of variations due to individual differences and noise. Therefore, normalization 1010 may be performed by the data preprocessing module 246 to ensure that the input data has a uniform scale. This step is crucial for the subsequent learning process to prevent features with larger scales from dominating the model's attention. That is, data scaling during the preprocessing stage may ensure that features extract from the EEG data have values in the same range and the features used in the underlying AI models are dimensionless. Scaling may be used for detecting outliers as well. Example data scaling techniques include normalization and standardization. In one embodiment, when the EEG data is scaled using normalization, the transformed data have a minimum and maximum value, with a default of 0 and 1. Standardization may be used when the EEG data is distributed according to the normal or Gaussian distribution. Standardized values may typically lie in the range [−2, 2], which represents the 95% confidence interval. Standardized values less than-2 or greater than 2 can be considered as outliers. Therefore, standardization can be used for outlier detection.

The normalized EEG data 1012 may be input into the detection phase 1004. A ConvLSTM model 1014 may be employed to analyze the normalized data 1012. ConvLSTM is a recurrent neural network (RNN) variant that incorporates convolutional structures in the LSTM units, making it well-suited for spatiotemporal data like EEG signals. Next, the anomaly detection module 248 may be configured to use the ConvLSTM model 1014 to process the data to differentiate between normal brain activity and potential seizures. It can detect two types of anomalies. A first type is point anomaly 1016 which refers to an individual data point that is significantly different from the rest of the data. In the context of EEG, this may indicate a sudden spike in electrical activity associated with a seizure. A second type is contextual anomaly 1018 which refers to anomalies that are context-specific, and may not be outliers if taken out of context. For EEG, this might involve unusual patterns that only indicate a seizure due to the temporal context they appear in. The ConvLSTM model 1014 may be configured to assess the correlation 1020 between the observed EEG patterns and known seizure patterns, assigning an anomaly score 1022 that quantifies how likely a segment of EEG data represents a seizure.

According to certain embodiments, abnormalities of neonatal EEG may be generally classified as follows: abnormalities of background rhythms, abnormalities of states and maturation, and neonatal seizures. One of the most striking features of the neonatal EEG is its discontinuity (periods of higher voltage activities followed by periods of lower ones that occur during portions of the recording). In the preterm neonate between 27 and 30 weeks of postmenstrual age, there may be a discontinuity of background rhythm compared to that of more mature newborns. For example, there may be high voltage bursts of fast activity interspersed with lower voltage activity. With maturity, the inter-burst intervals become shorter.

Further, the prolongation of inter-burst intervals (IBI) may be an indicator of diffuse brain injury or structural abnormality of the cortical network. No absolute criteria currently exist that can be used to determine whether records are excessively discontinuous. Some recent studies provide some guidance. Acceptable duration of the longest IBI according to postmenstrual age may include: 26 weeks=46 seconds; 27 weeks=36 seconds; fewer than 30 weeks=30 to 35 seconds; 31 to 33 weeks=20 seconds; 34 to 36 weeks=10 seconds; 37 to 40 weeks=6 seconds.

Processed neonatal EEG signals may indicate abnormal voltage and lack of differentiation of background activity. For example, a loss of faster frequencies in background activity is the earliest change in response to abnormalities in cortical function. Episodic generalized or regional voltage attenuation that is transient shows milder dysfunction compared to persistent, prolonged depression. The complete absence of poly frequency activity suggests a lack of differentiation along with other abnormalities and can be related to a severe brain insult. These can be noted in diffuse processes such as perinatal hypoxia-ischemia, metabolic encephalopathies, meningitis, encephalitis, cerebral and intraventricular hemorrhages. Persistently depressed and poorly differentiated activity in follow-up EEG recording after the first 24 hours carries a poor prognosis.

Disordered maturational development is referred to as “dyschronism” and, if present, is an important abnormality of the neonatal EEG. Dyschronism signifies a lag of maturity of the EEG from the expected postmenstrual age. The term external dyschronism refers to disordered maturational development where the features of EEG signals in all states demonstrate a lag or discrepancy between stated postmenstrual age (PMA) and the PMA estimated by a review of the EEG. A gap of greater than 3 weeks suggests significant neurological dysfunction, particularly if associated with an attenuation of background activity and multifocal epileptiform activity (sharp waves and spikes).

On the other hand, internal dyschronism refers to the discrepancy in the postmenstrual age determined during waking and in the phase of deeper stages of quiet sleep (QS). If a lag or discrepancy of more than 3 weeks is noted to be present, then this may also be taken as a marker of cerebral dysfunction.

Synchronous bursts (0.5 to 10 second duration) alternating with or followed by isoelectric background activity (2 to 10 seconds) in a processed EEG signal that is invariant and non-reactive is considered as fulfilling criteria for a suppression-burst pattern. This pattern can be seen in severe cerebral anoxia, drug-induced, toxic-metabolic encephalopathies, and severe structural malformations of the brain.

It is also the hallmark of epileptic encephalopathies with suppression-burst (Ohtahara syndrome and early myoclonic encephalopathy). In these conditions, the bursts contain very high amplitude polymorphic variable sharp and spike-wave patterns with or without symmetry, with low amplitude intervals that vary from 2 to 10 seconds, and the pattern persists in all stages of wakefulness and sleep.

Discontinuous EEG signals that do not meet the full criteria for burst suppression may be encountered more commonly, particularly in the context of perinatal hypoxic-ischemic encephalopathy. The bursting activity may be of longer duration (10 to 30 seconds), and IBI occupied by very low amplitude activity of less than 10 microV, less than 10-second duration. These features may be associated with loss of sleep cycling and lability.

There may be some EEG signal patterns of uncertain significance clinically. For example, positive temporal sharp waves (less than 400 ms in duration) seen over temporal (T3, T4) electrodes can also be encountered in term neonates and may not be considered to be of clinical significance unless persistent in serial records. Similarly, short-duration central sharp waves (3 to 7 seconds) may occur in short bursts and may not stand out from the background activity and are of uncertain clinical significance.

Focal/unilateral attenuation or voltage depression may present in the context of a persistent voltage asymmetry throughout recorded EEG signals. Focal infarcts, hemorrhage, cystic lesions, accumulation of subdural fluid, and sometimes scalp edema on the dependent side depending on the infant's position can be associated with such a focal or unilateral voltage depression.

Surface positive sharp waves that are broad-based, often localizing around C3, C4, and Cz electrodes and may occur at a frequency of 1 to 2 per min under pathological conditions. These waves are of higher amplitude and stand out against the background activity, and can sometimes be accompanied by superimposed waves of varying morphology and frequency. These PRSWs have been shown to be most frequently associated with periventricular white matter injury and consequential later motor impairments but have also been encountered in a variety of conditions such as meningitis, hydrocephalus, perinatal hypoxic-ischemic encephalopathy (HIE). They may be distinguished from sharp transients that can be encountered over similar locations but are of smaller amplitude and are less prominent.

Focal sharps and spikes may carry certain degrees of clinical significance based on the morphology, polarity, frequency or recurrence rate, and persistence in terms of regional expression. This applies to both temporal and extra-temporal expression of focal epileptiform activity. Persistent focal sharps often may be associated with a focal brain injury, while diffuse brain injury can be associated with multifocal expression of sharps or spikes. These findings may or may not be associated with identifiable abnormalities on imaging.

In terms of the correlation between neonatal seizures and EEG signals, neonatal seizures may be classified into: clinical seizures with electrographic correlation (electroclinical seizures); purely electrographic seizures (EEG expression of ictal rhythms without clinical correlation); and clinical only seizures (ictal behaviors without EEG correlates). In infants suspected of having seizure activity, those with abnormal EEG background are more likely to have seizures than those with a normal background. On the other hand, those with documented clinical seizure activity have been more likely to have a normal EEG background. Severe background EEG abnormalities such as burst suppression have been associated with early myoclonic encephalopathy (EME) and Ohtahara syndrome (Early Infantile Epileptic Encephalopathy). A theta pointu alternant pattern (generalized sharply contoured theta alternating with periods of generalized attenuation) has been reported in association with benign familial neonatal seizures as well as during the postictal state.

Sustained rhythmic activity on the EEG, with highly variable morphology (spikes, sharps, slow waves, mixed waveforms) amplitude and frequency, lasting for longer than 10 seconds, may indicate electrical seizure activity. Such activity may be rare under 33 to 34 weeks conceptional age; with increasing maturity of the neonatal brain, the ability to initiate and sustains seizure activity seems to become apparent. Another term, BIRDs (brief ictal rhythmic discharges), have been used to describe the rhythmic ictal activity of less than 10-second duration. Ictal rhythms are generally focal in onset, can arise in more than one region, tend to spread as the seizure evolves, often to one hemisphere, or may become generalized. The morphology of the discharge can change during a seizure, which helps differentiate it from monomorphic rhythmic patterns that may arise from artifacts. Specialized forms of focal discharges such as lateralized periodic discharges (LPDs) may be seen in neonates and have been described in association with herpes simplex encephalitis, focal ischemic strokes, and have sometimes been considered as indicative of seizures in a depressed brain.

Generalized ictal rhythms may be rare but may be seen in the context of generalized myoclonus (generalized sharps) and spasms (generalized attenuation or electro-decrement). In addition, electro-clinical dissociation or decoupling denotes a situation where in the context of the administration of automated external defibrillator (AED) to infants with electro-clinical seizures; the clinical manifestations may stop while the electrical ictal rhythms continue to be expressed. In another example, a sick infant is paralyzed for intubation and mechanical ventilation, there may be no clinical features, but the recorded EEG signal continues to show ictal rhythmic patterns.

The machine learning/deep learning model(s) 244 of the computing server 114 may use the anomaly detection module 248 to analyze segments of EEG data to locate one or more point anomalies 1016 by identifying waveforms (epileptiform discharges) that have a high and manifest degree of spike, and slow-wave complexes. Sharp wave transients are characterized by their negative or positive polarity, duration, abundance, spatial distribution, and repetitive behavior. A negative sharp wave transient has an initial and predominant deflection that is surface negative. A positive sharp wave transient has an initial and predominant deflection that is surface positive. Both need to be clearly distinct from the background as separate transients and not just sharply contoured background activity. Sharp wave transients lasting less than 100 msecs may be identified as spikes. Sharp wave transients lasting 100-200 msecs may be identified as sharp waves.

In another aspect, feature extraction of the EEG signals may involve segmenting data into smaller windows to have similar significant features for analysis purposes. The duration of these windows for epilepsy analysis may range from 5 to 60 seconds. One example implementation may use 20 seconds with overlap processes or a 5-second window without overlap.

Thereafter, the machine learning/deep learning model(s) 244 may be configured to determine the correlation between the observed EEG patterns and known seizure patterns in identifying the contextual anomaly 1018. In some aspects, certain factors including the patient's age, level of physical activity, mental state, level of consciousness, the influence of different biological factors, environmental factors, and pharmacological agents that can potentially influence the morphology of the waveforms may need to be taken into consideration by the machine learning/deep learning model(s) 244. There is a wide variation in the EEG waveforms. The contextual anomaly 1018 may be configured to identify differential normal or benign variants from pathologic EEG waveforms. Example normal variants may include but not limited to wicket spikes (e.g., waveforms appear over the temporal (anterior or mid-temporal) region during relaxed wakefulness, drowsiness, or state of light sleep); benign epileptiform transients of sleep (also called as small sharp spikes and occur in stage 1 or stage 2 of sleep); 6 Hz phantom spike-and-wave complex (a version of 3 Hz spike and wave pattern and the waveforms have low amplitudes and appear in the frequency of 5 Hz to 7 Hz); rhythmic mid-temporal theta of drowsiness (psychomotor variant) (this pattern is usually located in the mid temporal region and appears in relaxed wakefulness and drowsiness); positive occipital sharp transients of sleep (waveforms appear in the occipital regions during non-rapid eye movement sleep and are asymmetrically distributed); subclinical rhythmic EEG discharge in adults (benign EEG pattern which resembles ictal discharges and is, at times, interpreted as such, leading to a misdiagnosis of epilepsy); 14 Hz and 6 Hz positive spikes (6 and 14 Hz positive spikes are typically seen in the younger age group); repetitive vertex waves, especially in children; breach rhythm (waveforms seen over the regions that have a skull defect. Because of the skull defect, there is increased visibility of faster frequencies that are otherwise less appreciated on scalp EEG.).

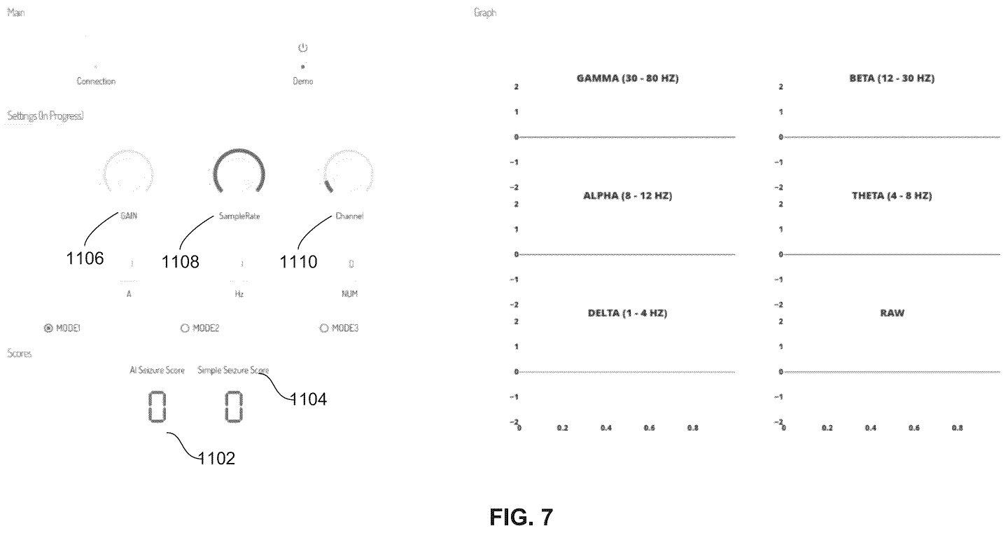

In some aspects, the machine learning/deep learning model(s) 244 of the computing server 114 may also include a training module 250 configured to improve seizure prediction accuracy of the artificial intelligence models used by the computing server 114. In one example, 835 hours of raw EEG data containing 1389 seizures recorded in a selected NICU of a hospital were used as datasets. In yet another embodiment, the training module 250 may be configured to predict and detect seizures based on raw EEG signals relating to a specific patient, thereby providing an individualized adaptive machine learning model with a higher accuracy of the machine learning model because it is based on personalized data from the user instead of generic data from an accumulation of other individuals. Therefore, automated model training allows for better seizure prediction and detection of a specific patient instead of an identification of seizure events determined based on data relating to a patent group. In yet another embodiment, the machine learning/deep learning model(s) 244 may be configured to compare the seizure detection results of the underlying AI models based on the performance criteria of the training module 250 with that of other detection algorithms used by computing server 114. In one embodiment, such comparison results may be displayed to a user of computing device 202 via the GUI 232. For example, referring to FIG. 7, the GUI 232 may be configured to display a first seizure score 1102 calculated based on AI technology and a second seizure score 1104 calculated based on other detection algorithms to a user. FIG. 7 also shows the raw EEG signals and processed EEG signals in each of a plurality of frequency ranges of interest (e.g., delta, theta, alpha, beta, and gamma waves) for seizure detection purposes. In one implementation, a user may also adjust the gain 1106, sample rate 1108, and channel selection 1110 via the GUI 232.

During the evaluation phase 1006, the performance of the machine learning/deep learning model(s) 244 may be determined using several metrics such as accuracy 1024 (the proportion of total predictions that were correct), recall 1026 (the ability of the model(s) to detect all relevant cases of seizures (true positives)), precision 1028 (the number of true positive predictions divided by the number of true positive and false positive predictions), F1-score 1030 (the harmonic mean of precision and recall, providing a single score that balances both concerns, and loss rate 1032 (the measure of prediction error; a lower loss rate indicates better model performance).

The ConvLSTM-based architecture, as shown in FIG. 6, may be designed to handle the complexity and variability of EEG data for effective seizure detection. By transforming the raw EEG signals into a normalized format and employing AI based model(s) that can capture both spatial and temporal relationships, system 100 of the present disclosure provides a reliable and accurate seizure detection of neonates, which is critical for timely medical intervention. For example, the training module 250 may be programmed and the underlying AI model(s) may be trained using a dataset of EEG recordings from multiple subjects, with labels indicating the occurrence of seizures. After training, the AI model's performance may be validated using additional data, and if the AI model meets selected criteria, it may be deployed in clinical settings to assist in real-time monitoring of patients for seizure detection.

Further, the notification generation module 252 of the computing server 114 may be configured to generate and transmit one or more text, visual and/or audio notifications to e.g., a doctor, a nurse, a family member or caregiver of a neonate, an epileptologist, a technician, etc. in response to detecting that the EEG data is indicative of a seizure that has, will, or is occurring in the neonate.

In some implementations, the seizure detection accuracy rate of the machine learning/deep learning model(s) 244 of the present disclosure may be configured to address the issue of imbalanced learning and imbalanced datasets. Certain standard classification learning algorithms may often be biased towards majority classes and therefore there is a higher misclassification rate in minority class instances. In other words, there may be a discrepancy between high accuracy and low recall in seizure detection when training dataset is imbalanced, which is common in medical datasets where normal readings are far more prevalent than anomalous seizure events. In one aspect, computing server 114 of the present disclosure may utilize more balanced sampling techniques or implement cost-sensitive learning which prioritizes the correct classification of the minority class. Moreover, model refinement of the machine learning/deep learning model(s) 244 may be carried out. This may include experimenting with various model architectures, adjusting hyperparameters, and innovating in feature engineering, all aimed at boosting the model's recall metric without unduly affecting its precision. Additionally, the integration of supplementary patient data and a broader array of EEG features may provide the AI model of the present disclosure with a richer context, aiding in the differentiation between seizure activities and normal brain patterns. Collaborating closely with clinical professionals may offer invaluable insights. Their expertise on the real-world consequences of false negatives and false positives in seizure detection can inform more nuanced adjustments to the AI model of the present disclosure, ensuring that it aligns with clinical needs and maximizes patient safety. For example, computing server 114 may use one of the data sources or services 116a, 116b, 116c, . . . 116n of FIG. 1 which may comprise an expert or knowledge based diagnostic or evaluation system for providing or optimizing interpretations of EEG signals that may include text, audio, video, and other rich media explanations. This comprehensive strategy aims not only to refine the model's technical accuracy but also to ensure its practical efficacy in a clinical setting.

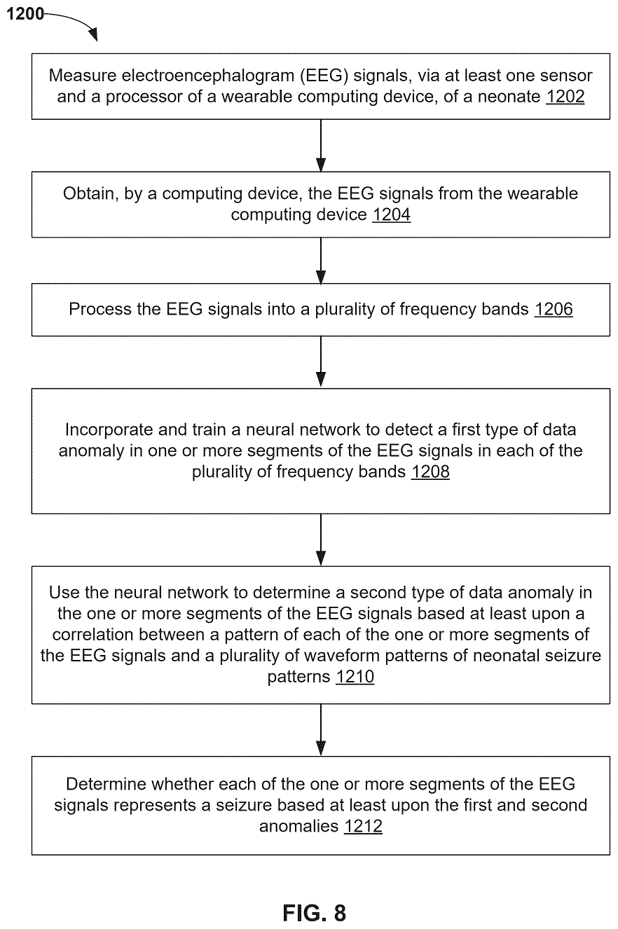

According to aspects of the present disclosure, FIG. 8 illustrates a flowchart of a method 1200 for monitoring and analyzing brain activity of neonates using advanced machine learning and deep learning techniques. Method 1200 may comprise measuring (1202) EEG signals of a neonate using a wearable computing device. In a preferred embodiment, the wearable computing device may be a headband enclosing at least one sensor placed on a selected location of the neonate's head to monitor brain activity of the neonate, and a processor configured to obtain the EEG signals measured by the at least one sensor. Method 1200 further comprises obtaining (1204) by, a computing device, the EEG signals from the wearable computing device, processing (1206) the EEG signals into a plurality of frequency bands, and incorporating and training (1208) a neural network to detect a first type of data anomaly in one or more segments of the EEG signals in each of the plurality of frequency bands. In addition, method 1200 comprises using (1210) the neural network to determine a second type of data anomaly in the one or more segments of the EEG signals based at least upon a correlation between a pattern of each of the one or more segments of the EEG signals and a plurality of waveform patterns of neonatal seizure patterns, and determining (1212) whether each of the one or more segments of the EEG signals represents a seizure based at least upon the first and second anomalies.

It should be appropriated that although various aspects and embodiments disclosed above are related to detecting neonatal seizures, the devices, systems and methods of the present disclosure may be used for infants, small children, and the elderly, or anyone who is unconscious and/or has detectable abnormal brain activity or patterns associated with neurological disorders. Further, the devices, systems and methods of the present disclosure may be used in a wide variety of settings such as home use or hospital use.

Various benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated.

1. A system deployed within a communication network for automatically detecting neonatal seizures, the system comprising:
a wearable computing device, comprising:
a headband enclosing at least one sensor placed on a selected location of a neonate's head to monitor brain activity of the neonate, and a first processor configured to obtain electroencephalogram (EEG) signals measured by the at least one sensor; and
a computing device, comprising:
a non-transitory computer-readable storage medium storing instructions, and
a second processor coupled to the non-transitory computer-readable storage medium and configured to execute the instructions to:
obtain the EEG signals from the wearable computing device,
process the EEG signals into a plurality of frequency bands,
incorporate and train a neural network to detect a first type of data anomaly in one or more segments of the EEG signals in each of the plurality of frequency bands,
use the neural network to determine a second type of data anomaly in the one or more segments of the EEG signals based at least upon a correlation between a pattern of each of the one or more segments of the EEG signals and a plurality of waveform patterns of neonatal seizure patterns, and
determine whether each of the one or more segments of the EEG signals represents a seizure based at least upon the first and second anomalies.

2. The system of claim 1, wherein the processor of the computing device is further configured to execute the instructions to calculate a score to quantify a likelihood of the one or more segments of the EEG signals represent a seizure of the neonate, and display the score via a graphical user interface of the computing device.

3. The system of claim 1, wherein the processor of the computing device is configured to execute the instructions to communicate with the wearable computing device via Bluetooth Low Energy (BLE) protocols.

4. The system of claim 1, wherein the processor of the computing device is configured to execute the instructions to process the EEG signals into the plurality of frequency bands using fast Fourier transform and normalize processed EEG signals to have a uniform scale.

5. The system of claim 1, further comprising a computing server deployed within the communication network and configured to host the neural network, wherein the neural network includes a Convolutional Long Short-Term Memory (ConvLSTM) model.

6. The system of claim 1, wherein the first type of data anomaly includes sharp waves and spikes in one or more segments of the EEG signals in each of the plurality of frequency bands, and the second type of data anomaly includes unusual EEG signal patterns indicating a seizure event due to a temporal context they appear in.

7. The system of claim 1, wherein the headband of the wearable computing device further encloses an accelerometer configured to track and analyze movements of the neonate, and a pulse oximeter configured to detect oxygen saturation and respiratory pattern changes associated with seizures in the neonate, and the processor of the computing device is further configured to execute the instructions to receive accelerometer and pulse oximeter data from the wearable computing device, and determine whether each of the one or more segments of the EEG signals represents the seizure based upon the accelerometer and pulse oximeter data.

8. A computer-implemented method, comprising:
measuring, by at least one sensor and a first processor of a wearable computing device, electroencephalogram (EEG) signals of a neonate;
obtaining, by a computing device, the EEG signals from the wearable computing device;
processing the EEG signals into a plurality of frequency bands;
incorporating and training a neural network to detect a first type of data anomaly in one or more segments of the EEG signals in each of the plurality of frequency bands;
using the neural network to determine a second type of data anomaly in the one or more segments of the EEG signals based at least upon a correlation between a pattern of each of the one or more segments of the EEG signals and a plurality of waveform patterns of neonatal seizure patterns; and
determining whether each of the one or more segments of the EEG signals represents a seizure based at least upon the first and second anomalies.

9. The computer-implemented method of claim 8, further comprising:
calculating a score to quantify a likelihood of the one or more segments of the EEG signals represent a seizure of the neonate; and
displaying the score via a graphical user interface of the computing device.

10. The computer-implemented method of claim 8, further comprising communicating, by the computing device, with the wearable computing device via Bluetooth Low Energy (BLE) protocols.

11. The computer-implemented method of claim 8, wherein the processing the EEG signals into the plurality of frequency bands comprises using fast Fourier transform to process the EEG signals in each of the plurality of frequency bands and normalizing processed EEG signals to have a uniform scale.

12. The computer-implemented method of claim 8, further comprising hosting, by computing server deployed within the communication network, the neural network, wherein the neural network includes a Convolutional Long Short-Term Memory (ConvLSTM) model.

13. The computer-implemented method of claim 8, wherein the first type of data anomaly includes sharp waves and spikes in one or more segments of the EEG signals in each of the plurality of frequency bands, and the second type of data anomaly includes unusual EEG signal patterns indicating a seizure event due to a temporal context they appear in.

14. The computer-implemented method of claim 8, further comprising:
tracking and analyzing movements of the neonate using an accelerometer enclosed in the headband of the wearable computing device;
detecting oxygen saturation and respiratory pattern changes associated with seizures in the neonate using a pulse oximeter;
receiving, by the computing device, accelerometer and pulse oximeter data from the wearable computing device; and
determining whether each of the one or more segments of the EEG signals represents the seizure based upon the accelerometer and pulse oximeter data.

15. A non-transitory computer readable medium storing machine executable instructions for a system deployed within a communication network for automatically detecting neonatal seizures, the machine executable instructions being configured for:
measuring, by at least one sensor and a first processor of a wearable computing device, electroencephalogram (EEG) signals of a neonate;
obtaining, by a computing device, the EEG signals from the wearable computing device;
processing the EEG signals into a plurality of frequency bands;
incorporating and training a neural network to detect a first type of data anomaly in one or more segments of the EEG signals in each of the plurality of frequency bands;
using the neural network to determine a second type of data anomaly in the one or more segments of the EEG signals based at least upon a correlation between a pattern of each of the one or more segments of the EEG signals and a plurality of waveform patterns of neonatal seizure patterns; and
determining whether each of the one or more segments of the EEG signals represents a seizure based at least upon the first and second anomalies.

16. The non-transitory computer readable medium of claim 15, further comprising instructions for:
calculating a score to quantify a likelihood of the one or more segments of the EEG signals represent a seizure of the neonate; and
displaying the score via a graphical user interface of the computing device.

17. The non-transitory computer readable medium of claim 15, further comprising instructions for communicating, by the computing device, with the wearable computing device via Bluetooth Low Energy (BLE) protocols.

18. The non-transitory computer readable medium of claim 15, wherein the instructions for processing the EEG signals into the plurality of frequency bands comprise instructions for using fast Fourier transform to process the EEG signals in each of the plurality of frequency bands and normalizing processed EEG signals to have a uniform scale.

19. The non-transitory computer readable medium of claim 15, further comprising instructions for:
hosting, by computing server deployed within the communication network, the neural network, wherein the neural network includes a Convolutional Long Short-Term Memory (ConvLSTM) model;
tracking and analyzing movements of the neonate using an accelerometer enclosed in the headband of the wearable computing device;
detecting oxygen saturation and respiratory pattern changes associated with seizures in the neonate using a pulse oximeter;
receiving, by the computing device, accelerometer data from the wearable computing device; and
determining whether each of the one or more segments of the EEG signals represents the seizure based upon the accelerometer data.

20. The non-transitory computer readable medium of claim 15, wherein the first type of data anomaly includes sharp waves and spikes in one or more segments of the EEG signals in each of the plurality of frequency bands, and the second type of data anomaly includes unusual EEG signal patterns indicating a seizure event due to a temporal context they appear in.
